(19) United States
(12) Patent Application Publication
(10) Pub. No.: US 2026/0165748 A1
(43) Pub. Date: Jun. 18, 2026

(54) PIVOTAL BONE ANCHOR ASSEMBLIES WITH TWO-PIECE RECEIVERS, SEPARATE RETAINING STRUCTURES, AND BI-SPHERIC SHANK HEADS

(71) Applicant: Roger P. Jackson, Prairie Village, KS (US)

(72) Inventors: Roger P. Jackson, Prairie Village, KS (US); James L. Surber, Kansas City, KS (US); Nathaniel D. Ginzton, Boise, ID (US)

(21) Appl. No.: 19/422,363

(22) Filed: Dec. 16, 2025

Related U.S. Application Data
(60) Provisional application No. 63/734,705, filed on Dec. 16, 2024.

(51) Int. Cl. A61B 17/70

(52) U.S. Cl. CPC A61B 17/7037; A61B 17/7032

(57) ABSTRACT

A pivotal bone anchor assembly includes a two-piece receiver comprising an upper body having an open channel for receiving a rod and an annular lower end with a downwardly-extending first connection structure, and a cylindrical base having a spherical seating surface adjacent a bottom opening and an upwardly-extending second connection structure configured for coupling with the first connection structure to secure the cylindrical base to the upper body. The assembly also includes a bone anchor comprising a shank head and an anchor portion, and a cap retainer having plurality of retainer collet fingers configured to resiliently expand to capture the shank head within the receiver. The assembly further includes a collet insert having an insert channel for engaging the rod and a plurality of insert collet fingers configured to resiliently expand to capture the cap retainer. The collet insert and cap retainer are together positionable within the upper body prior to the cylindrical base and the upper body being coupled together to complete the two-piece receiver.

### CROSS-REFERENCE TO RELATED APPLICATIONS

This application claims the benefit of U.S. Provisional Application No. 63/734,705, filed Dec. 16, 2024, which is incorporated by reference in its entirety herein and for all purposes.

### FIELD

The present disclosure relates generally to spinal implant assemblies utilizing universal shank heads having a common capture portion geometry, and that are configured for connection with an array or collection of receiver sub-assemblies having different functionalities, and their use in surgery involving vertebral body stabilizations with spinal fixation systems.

### BACKGROUND

Spinal implants in general, and bone anchors or screws in particular, are used in many types of spinal surgery in order to secure various implants to vertebrae along the spinal column for the purposes of treating spinal disorders, such as degenerative conditions and deformities, and also for stabilizing and/or adjusting spinal alignment. A common mechanism for providing vertebral support is to implant the bone screws into certain bones which then, in turn, support a longitudinal structure such as an elongate rod, or are supported by such a rod. Although both closed-ended and open-ended spinal implants, such as bone screws and hooks, are known, the open-ended spinal implants can be particularly well suited for connections to rods and connector arms because such rods or arms do not need to be passed through a closed bore, but rather can be laid or urged into an open channel within the head or receiver of such a screw, hook, or connector. For example, open-ended bone screws generally comprise an anchor portion, such as a threaded shank, connected to a head or receiver having a pair of upwardly-projecting branches or arms which form a yoke that defines a slot or channel configured to receive the rod. The slot or channel could have different shapes, such as, a U-shape or a square shape. Moreover, the threaded shanks of the bone screws can also be replaced with hooks or other types of bone anchors or connectors to form a variety of different types of spinal implants, also having open ends for receiving rods or portions of other structures, and wherein such implants can facilitate surgical techniques performed with different spinal fixation systems.

Early bone screws used in spinal surgery generally had a yoke-shaped ‘head’ that was integrally formed or “fixed” with the threaded shank, and therefore immovable. Because the fixed head could not be moved relative to the shank, these fixed bone screws needed to be favorably positioned in the spine. Otherwise, the elongate rod would need to be bent in order for it to be placed within the rod-receiving channels of multiple implants due to their alignment. Given the highly curved shape of the spines of some patients, however, this is sometimes very difficult or impossible to do. Therefore, polyaxial (i.e., multiplanar), uni-planar (i.e., monoplanar), and/or translatable pivotal bone screws or bone anchor assemblies, were developed and are now commonly preferred. Open-ended polyaxial bone screw assemblies typically allow for pivoting and rotation of the connected but completely separate yoke-shaped receiver or receiver sub-assembly about an enlarged spherical ‘head’ or upper capture portion of the threaded shank or bone anchor in one or more planes, until a desired rotational and pivotal position of the receiver is achieved relative to the shank. This can be accomplished by manipulating the position of the receiver relative to the shank during a final stage of a medical procedure when the elongate rod or other longitudinal connecting member is inserted into the receiver or receiver sub-assembly, followed by a locking set screw, a plug, a closure, or other type of locking mechanism known in the art.

It is understood that spinal fixation systems generally include a variety of components that require some assembly, such as the various types of bone anchors, the rods or connector arms, and the closures or plugs with the receivers or receiver sub-assemblies, with each component having specific features with respect to structure and function. Moreover, the receiver sub-assemblies can further include components in addition to the receiver itself, such as pressure inserts, spring rings, separate retainers, and other components of different types that are operable to connect these receiver sub-assemblies with the heads of the bone anchors. The pressure inserts, rings, retainers, and other components can be pre-assembled together within the receivers to form the receiver sub-assemblies that are ready for further assemblage with the bone anchors, and eventually with the rods or connector arms and the closures or plugs.

Some designs provide for the threaded shanks or other types of bone anchor to be bottom loaded into the receiver sub-assemblies. With bottom loaded bone anchor assemblies, for example, some designs known in the art require a separate retainer to hold the shank within the receiver, with the receiver having a bottom opening large enough to allow for the head or upper capture portion of the threaded shank or bone anchor to be uploaded into the central bore or cavity of the receiver. Other types of bottom loaded bone anchor assemblies do not include the separate retainer, however, and instead include a receiver having a lower portion with a bottom opening that is configured to directly threadably mate with the head or upper capture portion of the shank that can be configured as a threaded spherical head to provide for polyaxial or multiplanar motion.

Further to the above, bottom loaded bone anchor assemblies can also be fully assembled by the spinal company or distributor before being shipped to a hospital, so as to help with inventory management, or can be shipped as a modular array of multiple separate and different shanks and a fewer number of pre-assembled receiver sub-assemblies that can then be fully assembled, for example, at the hospital or surgical center during a surgery, thereby saving costs. Additionally, the modular spinal implants can be fully assembled at the hospital either before insertion into the patient, or after the threaded shank or bone anchor has been inserted into the patient, such as with robotic assistance or directly by a robot. The different techniques or approaches for the insertion and assembly of the modular parts of the bone anchor assemblies can be described as ex-vivo and in-vivo, respectively.

### SUMMARY

The present disclosure is generally directed to bone anchor assemblies that include a two-piece receiver comprising an upper body having an open channel for receiving a rod and an annular lower end with a downwardly-extending first connection structure, and a circular base having a spherical seating surface adjacent a bottom opening and an upwardly-extending second connection structure configured for coupling with the first connection structure to secure the circular base to the upper body. The assemblies also include bone anchors comprising a capture portion, such as a universal or bi-spheric shank head, and an anchor portion opposite the capture portion configured for attachment to the bone of a patient. The assemblies further include retaining structures or cap retainers configured to secure the capture portion within the circular base, and collet inserts having an upper surface configured to receive the rod and a lower collet portion configured to receive and capture the cap retainers. In each embodiment of the bone anchor assemblies the cap retainer and the collet insert are positionable together into the upper body prior to the circular base and the upper body being coupled together to complete the two-piece receiver and receiver sub-assembly.

Additional embodiments of the present disclosure will be better understood upon review of the detailed description set forth below taken in conjunction with the accompanying drawing figures, which are briefly described as follows.

### BRIEF DESCRIPTION OF THE DRAWINGS

FIG. 1 is an exploded perspective view of a multiplanar bone anchor assembly, in accordance a representative embodiment of the present disclosure.

FIG. 2 is a perspective view of the bone anchor of the multiplanar bone anchor assembly of FIG. 1.

FIG. 3 is a close-up perspective view of the capture portion of the bone anchor of FIG. 2.

FIG. 4 is a cross-sectional view of the of the bone anchor of FIG. 2.

FIG. 5 is a close-up cross-sectional view of the capture portion of the bone anchor of FIG. 2.

FIG. 6 is a perspective view of the upper body of the two-piece receiver of the multiplanar bone anchor assembly of FIG. 1.

FIG. 7 is a top view of the upper body of FIG. 6.

FIG. 8 is a cross-sectional side view of the upper body of FIG. 6.

FIG. 9 is another cross-sectional side view of the upper body of FIG. 6.

FIG. 10 is a perspective view of the circular base of the two-piece receiver of the multiplanar bone anchor assembly of FIG. 1.

FIG. 11 is a cross-sectional view of the circular base of the two-piece receiver of FIG. 10.

FIG. 12 is an exploded cross-sectional view of the two-piece receiver of the multiplanar bone anchor assembly of FIG. 1 prior to the assembly of the circular base to the upper body.

FIG. 13 is a cross-sectional view of the assembled two-piece receiver of FIG. 12 after the assembly of the circular base to the upper body.

FIG. 14 is a perspective view of the cap retainer of the multiplanar bone anchor assembly of FIG. 1.

FIG. 15 is a cross-sectional perspective view of the cap retainer of FIG. 14.

FIG. 16 is a partially-sectioned perspective view of the cap retainer and bi-spheric shank head of the multiplanar bone anchor assembly of FIG. 1 showing the multiplanar shank head sub-assembly.

FIG. 17 is a partially-sectioned front view of the multiplanar shank head sub-assembly of FIG. 16.

FIG. 18 is a perspective view of the collet insert of the multiplanar bone anchor assembly of FIG. 1.

FIG. 19 is a bottom perspective view of the collet insert of FIG. 18.

FIG. 20 is a side view of the collet insert of FIG. 18.

FIG. 21 is a cross-sectional side view of the collet insert of FIG. 18.

FIG. 22 is an exploded partially-sectioned perspective view of the components of the multiplanar receiver sub-assembly prior to their pre-assembly into a shipping state configuration.

FIG. 23 is an exploded and partially-sectioned perspective view of the components of the multiplanar receiver sub-assembly of FIG. 22, showing the pre-assembly of the cap retainer with the collet insert prior to uploading together into the upper body of the two-piece receiver.

FIG. 24 is a partially-sectioned perspective view of the pre-assembled cap retainer with the collet insert after uploading together into the upper body of the two-piece receiver, prior to connecting the circular base with the upper body.

FIG. 25 is a partially-sectioned perspective view of the upper body and circular base after connection together around the pre-assembled cap retainer and collet insert to form a multiplanar receiver sub-assembly in the shipping state configuration.

FIG. 26 is a partially-sectioned front view of the multiplanar receiver sub-assembly of FIG. 25 in the shipping state configuration.

FIG. 27 is a partially-sectioned front view of the multiplanar receiver sub-assembly of FIGS. 25-26 being assembled with the bi-spheric shank head of the universal bone anchor, with the bi-spheric shank head moving upward through the bottom opening of the receiver until the bi-spheric shank head engages the cap retainer that is suspended within the internal cavity of the receiver by the lower collet portion of the collet insert.

FIG. 28 is partially-sectioned front view of the multiplanar receiver sub-assembly as the bi-spheric shank head continues to drive upward as it expands the cap retainer within the lower collet portion of the collet insert until reaching the maximum expansion of the cap retainer.

FIG. 29 is a partially-sectioned front view of the multiplanar receiver sub-assembly as the bi-spheric shank head continues to drive upward until the bi-spheric shank head is completely captured by the cap retainer to form the multiplanar shank head sub-assembly secured within the multiplanar receiver sub-assembly.

FIG. 30 is a partially-sectioned front view of the multiplanar receiver sub-assembly after the collet insert has been downwardly deployed to push the multiplanar shank head sub-assembly back downward against the seating surface of the receiver to establish the multiplanar bone anchor assembly in a pre-lock friction fit, and with the bone anchor in an articulated position.

FIG. 31 is a partially-sectioned perspective view of the multiplanar bone anchor assembly, fully assembled with the elongate rod and closure and with the bone anchor in an articulated position relative to the receiver.

FIG. 32 is another partially-sectioned perspective view of the fully-assembled multiplanar bone anchor assembly of FIG. 31, with the bone anchor in a different articulated position relative to the receiver.

FIG. 33 is an exploded perspective view of a turret-type multiplanar (i.e., turret) bone anchor assembly, in accordance another representative embodiment of the present disclosure.

FIG. 34 is a perspective view of the upper body of the two-piece receiver of the turret bone anchor assembly of FIG. 33.

FIG. 35 is a bottom perspective view of the upper body of FIG. 34.

FIG. 36 is a cross-sectional front view of the upper body of FIG. 34.

FIG. 35 is a cross-sectional side view of the upper body of FIG. 34.

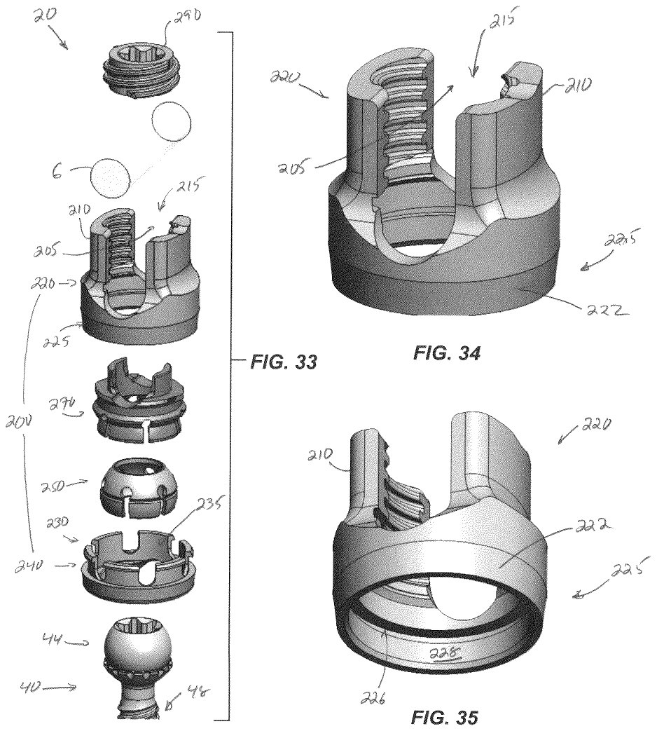

FIG. 38 is a perspective view of the circular base of the two-piece receiver of the turret bone anchor assembly of FIG. 33.

FIG. 39 is a bottom perspective view of the circular base of FIG. 38.

FIG. 40 is a cross-sectional front view of the assembled two-piece receiver of the turret bone anchor assembly of FIG. 33.

FIG. 41 is a perspective view of the collet insert of the turret bone anchor assembly of FIG. 33.

FIG. 42 is a bottom perspective view of the collet insert of FIG. 41.

FIG. 43 is a side view of the collet insert of FIG. 41.

FIG. 44 is a cross-sectional side view of the collet insert of FIG. 41.

FIG. 45 is a perspective view of the cap retainer of the turret bone anchor assembly of FIG. 33.

FIG. 46 is a partially-sectioned perspective view of the cap retainer and bi-spheric shank head of the turret bone anchor assembly of FIG. 33 assembled together to form the turret shank head sub-assembly.

FIG. 47 is a partially-sectioned front view of the components of the turret bone anchor assembly of FIG. 33, showing the pre-assembled cap retainer and collet insert being uploaded together into the upper body of the two-piece receiver.

FIG. 48 is a partially-sectioned front view of the components of FIG. 47, showing the pre-assembled cap retainer and collet insert after being uploaded together into the upper body of the two-piece receiver, prior to connecting the circular base with the upper body.

FIG. 49 is a partially-sectioned front view of the upper body and circular base after connection together around the pre-assembled cap retainer and collet insert to form a turret receiver sub-assembly in the shipping state configuration, with the turret receiver sub-assembly positioned above the bi-spheric shank head.

FIG. 50 is a partially-sectioned front view of the turret receiver sub-assembly after the collet insert has been downwardly deployed to form the turret anchor assembly in a pre-lock friction fit.

FIG. 51 is a perspective view of the turret bone anchor assembly fully assembled with the elongate rod and closure and with the bone anchor in an articulated position relative to the receiver.

FIG. 52 is a partially-sectioned front view of the fully-assembled turret bone anchor assembly of FIG. 51, with the bone anchor in a different articulated position relative to the receiver.

Those skilled in the art will appreciate and understand that the various features and structures or components of the bone anchor assemblies shown in the drawings described above, together with their relative relationships, interconnections and functions, can be interpreted as being drawn to scale. Nevertheless, it is also understood that the representative embodiments of the present disclosure disclosed and claimed herein are not limited to the precise structures and interrelationships of the features and components shown in the drawing figures, and that the dimensions, relative positions, and interconnections between the illustrated features and components may also be expanded, reduced, re-shaped, or otherwise revised or altered as needed to more clearly illustrate the structure of the embodiments depicted therein or the functions of the various features and components, as described below. Again, it is foreseen that some parts and features are interchangeable in their arrangement between the different embodiments disclosed.

### DESCRIPTION OF THE INVENTION

The following description, in conjunction with the accompanying drawings, is provided as an enabling teaching of bone anchors having a representative type of ‘universal’ shank head configured to cooperate with separate retaining structures that, in turn, have been pre-assembled together with collet inserts into receivers to form receiver sub-assemblies with different functionalities, and with the bi-spheric shank heads being bottom-loaded into the pre-assembled receiver sub-assemblies. As described below, the representative type of universal shank head or capture portion of a bone anchor illustrated herein is a bi-spheric shank head or capture structure comprising an upper partial spherical portion of lesser diameter that extends below a hemisphere plane, and a lower partial spherical portion of greater diameter that begins at a lower offset plane that is spaced below the hemisphere plane to extend downward and merge with the neck of the shank body, and with an upward-facing shelf or annular ledge extending between the upper partial spherical portion and the lower partial spherical portion.

The bone anchors are generally configured for use with a collection or array of complementary pivotal and non-pivotal receiver sub-assemblies in a spinal fixation system. In particular, the collection can include different types of receiver sub-assemblies that can be coupled to the bi-spheric shank heads of the bone anchors to form bone anchor assemblies having different and specialized modes of movement, degrees of freedom, or modalities (with the terms ‘mode’, ‘modality’, and ‘multi-modal’, etc., being used herein to describe the way in which something moves), including but not limited to pivoting and non-pivoting but axially rotatable (e.g. monoaxial) movement of the receiver sub-assembly relative to a shank or bone anchor that is further configured for implantation into the bone of a patient. The description also includes one or more methods for assembling and employing the bone anchors with the multi-modal collection of receiver sub-assemblies. As described below, individual bone anchor assemblies, systems, and/or methods of assembly and/or use of the present disclosure for this representative type of universal shank head can provide significant advantages and benefits over other pivotal and/or non-pivotal bone anchors and spinal fixation systems known in the art due to, in one aspect, the degree of versatility and adaptability provided by the shank head universality (i.e. all of the shank heads having a common geometry that is connectable with each type of receiver sub-assembly that has its own predetermined combination of degrees of freedom and operational functionalities). The recited advantages are not meant to be limiting in any way, however, as one skilled in the art will appreciate that other advantages and benefits may also be realized upon practicing the present disclosure.

Furthermore, those skilled in the relevant art will recognize that changes can be made to the disclosed embodiments for shank head universality, beyond those described, while still obtaining the beneficial results. It will also be understood and appreciated that some of the advantages and benefits of the described embodiment for the invention can be obtained by selecting some of the features (e.g., the structures or components) of the disclosed receiver sub-assemblies without utilizing other features, and that features from one sub-assembly embodiment may be interchanged or combined with features from other sub-assemblies in any appropriate combination. For example, any individual feature or collective features of method embodiments may be applied to apparatus, product or system embodiments, and vice versa. Likewise, structural elements or functional features from one embodiment may also be combined with or replaced by structural elements or functional features from one or more additional embodiments in any suitable manner. Those who work in the art will therefore recognize that many modifications and adaptations to the representative embodiments described herein are possible and may even be desirable in certain circumstances, and are to be considered part of the present disclosure. Thus, it will be appreciated that the present disclosure is provided as an illustration of the principles for the representative modular spinal fixation system incorporating the bi-spheric shank head that are shown and discussed therein, since the scope of each invention disclosed herein is to be defined by their respective claims.

### Multiplanar Bone Anchor Assembly

Referring now in more detail to the drawing figures, wherein like parts are identified with like reference numerals throughout the several views, FIG. 1 is an exploded perspective view of one representative embodiment of a multiplanar pivotal bone anchor assembly 10 that includes a bone anchor 50, such as a threaded shank, having a bi-spheric shank head or capture portion 60, and an anchor portion 84 opposite the bi-spheric shank head 60 configured for securement within or attachment to the bone of a patient.

The multiplanar bone anchor assembly 10 also includes a multiplanar two-piece receiver 100 or housing having a circular base 140 defining the lower portion of an internal cavity 135 that is configured to receive the capture portion 60 of the bone anchor 50, and an upper body 120 that includes a pair of upright arms 110 integrally formed with and extending upward from an annular lower end 125 to define an open rod channel 105 configured to receive an elongate rod 4. The annular lower end 125 and upright arms 110 can together define a central bore 115 that extends upward from the internal cavity 135 through the rod channel 105 to the tops of the upright arms 110, and which is centered about the vertical centerline axis 101 of the two-piece receiver 100.

The two-piece receiver 100 can be initially pivotably secured to the capture portion 60 of the bone anchor 50 with a number of separate internal components that have been pre-assembled into the internal cavity 135 and the central bore 115 to form a multiplanar receiver sub-assembly 12. These internal components can include, but are not limited to, a pivoting or articulating multiplanar cap retainer 150 that is ultimately positioned in the internal cavity 135 or lower portion of the central bore 115, and which attaches to the bi-spheric shank head 60 (see FIGS. 16-17) to pivotably couple the shank 50 to the receiver sub-assembly 12. The receiver sub-assembly 12 further includes a collet insert 170, also known as a compression element, which can be positioned above the cap retainer 150 in a middle portion of the central bore 115 where the central bore 115 intersects with the rod channel 106, and is operable to engage with the cap retainer 150 below and to be engaged by the elongate rod 4 from above. In one aspect the collet insert 170 can be downwardly-displaceable with a tool or tooling from an upper shipping-state position to a lower friction-fit position after the bi-spheric shank head 60 has been uploaded into the receiver sub-assembly, so as to establish a non-floppy pre-lock friction fit configuration prior to final assembly with the elongate rod and the closure.

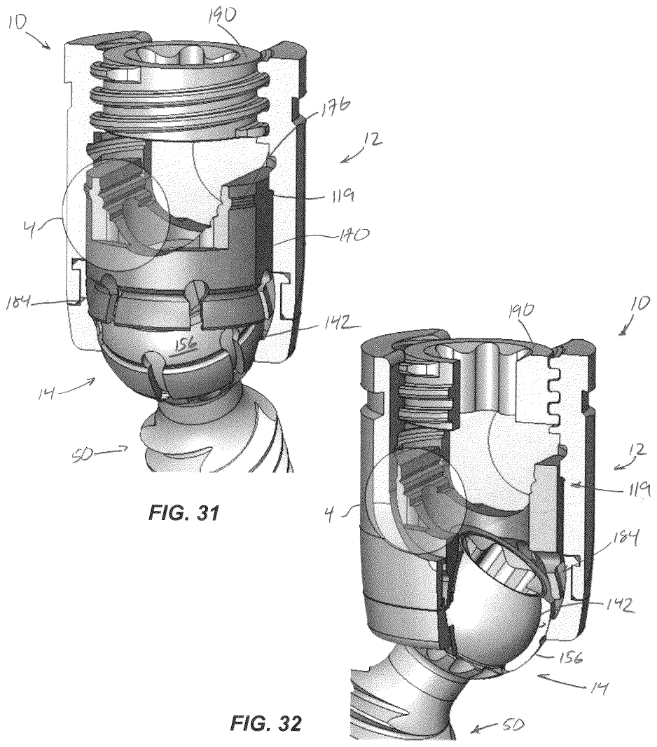

After the elongate rod 4 has been positioned within a lower portion of the rod channel and into engagement with the collet insert 170, a closure 190 can be threadably or otherwise secured into an upper portion of the central bore or rod channel to apply pressure to an upper surface of the rod 4, such as by direct contact, thereby locking both the elongate rod 4 and the multiplanar bone anchor assembly 10 into a final locked configuration or position, such as that shown in FIGS. 31-32. It is foreseen that the collet insert 170 can also be downwardly-displaceable from the shipping state position to the friction fit and/or locked position simultaneous with the placement of the rod and threaded installation of the closure.

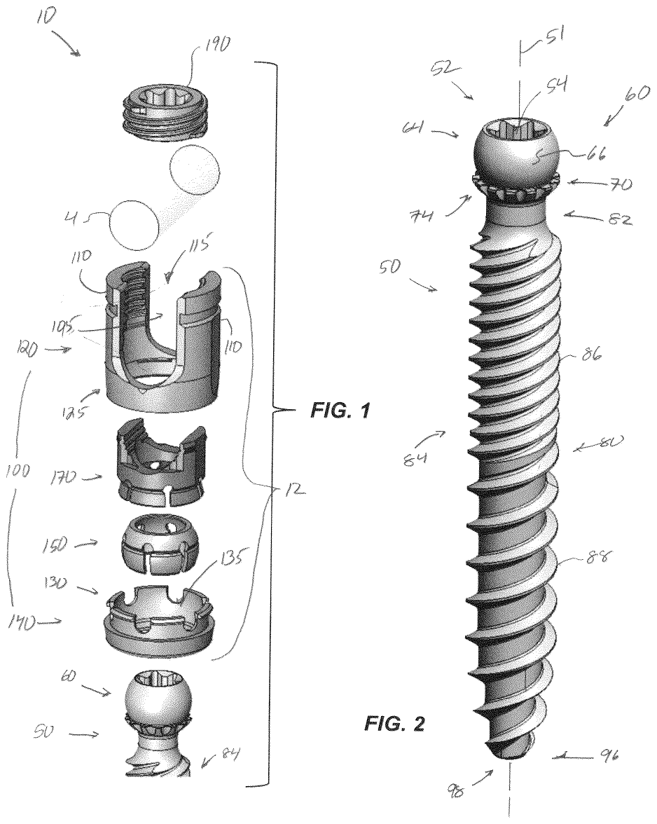

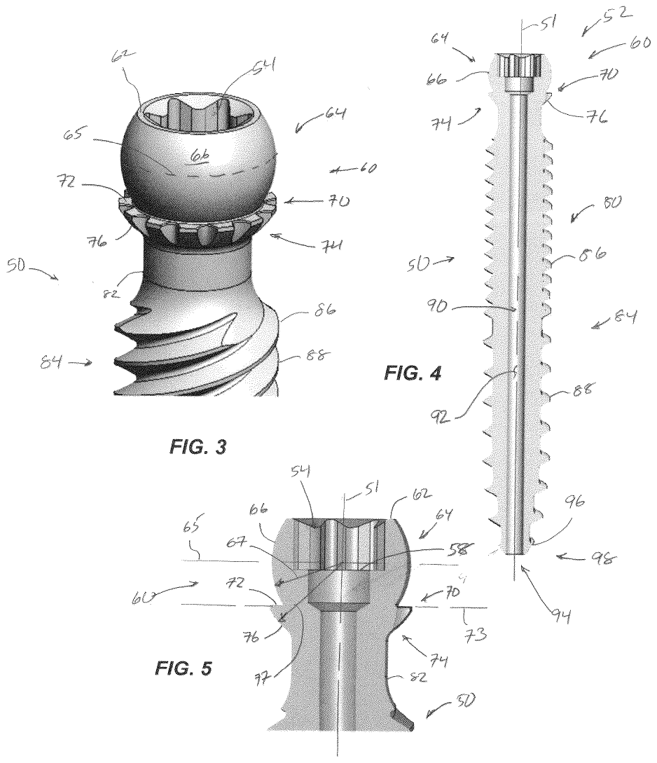

With reference to the exploded perspective view of FIG. 1 and the isolated views of FIGS. 2-5, the bone anchor 50 includes the bi-spheric shank head or capture structure 60 at an upper or proximal end 52, and a body 80 extending distally from the capture portion 60 with an attachment or anchor portion 84 at a distal end 98 configured for fixation to the bone of a patient. The body of the shank 80 can be integral with the bi-spheric shank head 60 and can include a neck portion or neck 82 that extends between the bi-spheric shank head 60 and the anchor portion 84. In one aspect the neck 82 can have a cross-sectional diameter that is less than both the diameter(s) of the bi-spheric shank head 60 and the cross-sectional diameter of the anchor portion 84 immediately below the neck 82, and can be configured to pivot against an inner edge of the lower opening of the receiver of the multiplanar bone anchor assembly 10 so as to provide an increased angle of articulation between the receiver and the bone anchor 50. As shown, the anchor portion 84 can be a threaded anchor portion with one or more bone engagement threads, such as a full length dual-lead thread form 88 extending the length of the body of the shank 80 from the distal tip 96 to the neck 82, and a partial length dual-lead thread form 86 beginning at an intermediate location and extending along an upper portion of the shank body 80 to the neck 82.

The bi-spheric shank head 60 at the proximal end 52 of the shank 50 generally comprises an upper partial spherical portion 64 defining an upper spherical surface 66 that extends above and below a hemisphere plane 64 of the bi-spheric shank head 60, and a lower partial spherical portion 74 defining a lower spherical surface 76 that begins at a lower offset plane 73 that is spaced below the hemisphere plane 65 to extend downward and merge with the neck 82 of the shank body 80. A lower upward-facing shelf or annular ledge 70 extends between the upper inner partial spherical portion 64 and the lower partial spherical portion 74, and can be considered the portion of the lower partial spherical portion 74 that extends radially outward beyond the upper spherical surface 66. As shown in the drawings, in one aspect the annular lower ledge 70 can define an upward-facing planar ledge surface 72 that extends perpendicular to the longitudinal axis 51 of the shank along the lower offset plane 73 between the upper and lower partial spherical portions. It is foreseen, nevertheless, that the lower ledge may not extend along the lower offset plane and may instead intersect the lower offset plane and the upper edge of the lower partial spherical portion at an acute angle, thereby defining a generally upward-facing ledge surface that is frusto-conical rather than planar, whether extending upwardly and outwardly, or downwardly and outwardly, from the upper partial spherical portion 64 to the lower partial spherical portion 74.

As described above, the top surface 62 of the capture portion 60 can be an annular planar top surface that surrounds an internal drive feature 54 or drive socket. The illustrated internal drive feature 54 is an aperture formed in the top surface 62, and in one aspect can be a multi-lobular or star-shaped aperture, such as those sold under the trademark TORX, or the like, having internal faces designed to receive a multi-lobular or star-shaped tool for rotating and driving the bone anchor 50 into the vertebra. It is foreseen that such an internal tool engagement structure or drive feature 54 may take a variety of tool-engaging forms and may include one or more apertures of various shapes, such as a pair of spaced apart apertures or a hex shape designed to receive a hex tool (not shown) of an Allen wrench type. The seat or base surface of the drive feature 54 can be disposed perpendicular to the longitudinal axis 51 of the shank 50, with the drive feature otherwise being coaxial with the longitudinal axis 51. In operation, a driving tool is received in the internal drive feature 54, being seated at the base surface and engaging the internal faces of the drive feature for rotating and driving the anchor portion 84 of the bone anchor into the vertebra, either before or after the bone anchor 50 is attached or coupled to the multiplanar receiver sub-assembly 12. If attached, the threaded anchor portion 84 of the body 80 of the bone anchor can be driven into the vertebra with the driving tool extending downward through both the central bore 115 of the multiplanar receiver 100 and the central aperture of the collet insert 170 of the multiplanar receiver sub-assembly 12.

In one aspect the bone anchor 50 or shank can be also cannulated (and also fenestrated for the application of bone cement, or even expandable) with a narrow axial bore 90 or aperture extending through the entire length thereof and centered about the longitudinal axis 51 of the shank 50. The axial bore 90 can be defined by an inner cylindrical sidewall 92 with a lower circular opening 94 at the distal end 98 of the shank body 80, and an upper circular opening 58 communicating with the internal drive socket 54 at the internal base surface thereof, and is coaxial with the body 80 and the capture portion 60 of the bone anchor 50. The axial bore 90 provides a passage through the shank interior for a length of wire (not shown) inserted into the vertebra prior to the implantation of the anchor portion 84 of the bone anchor 50, the wire providing a guide for insertion of the anchor portion 84 into the vertebra. The axial bore 90 can also provide for a pin to extend therethrough and beyond the distal end 98 of the shank body 80, the pin being associated with a tool to facilitate insertion of the body 80 of the bone anchor 50 into the vertebra.

To provide a biologically active interface with the bone, the body 80 of the bone anchor 50, including both the threaded anchor portion 84 and the neck 82, may be coated, perforated, made porous or otherwise treated or textured. The treatment may include, but is not limited to a plasma spray coating or other type of coating of a metal or, for example, a calcium phosphate; or a roughening, perforation or indentation in the shank surface, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca3(PO4)2, tetra-calcium phosphate (Ca4P2O9), amorphous calcium phosphate and hydroxyapatite (Ca10(PO9)6(OH)2). Coating with hydroxyapatite, for example, is desirable as hydroxyapatite is chemically similar to bone with respect to mineral content and has been identified as being bioactive and thus not only supportive of bone ingrowth, but actively taking part in bone bonding.

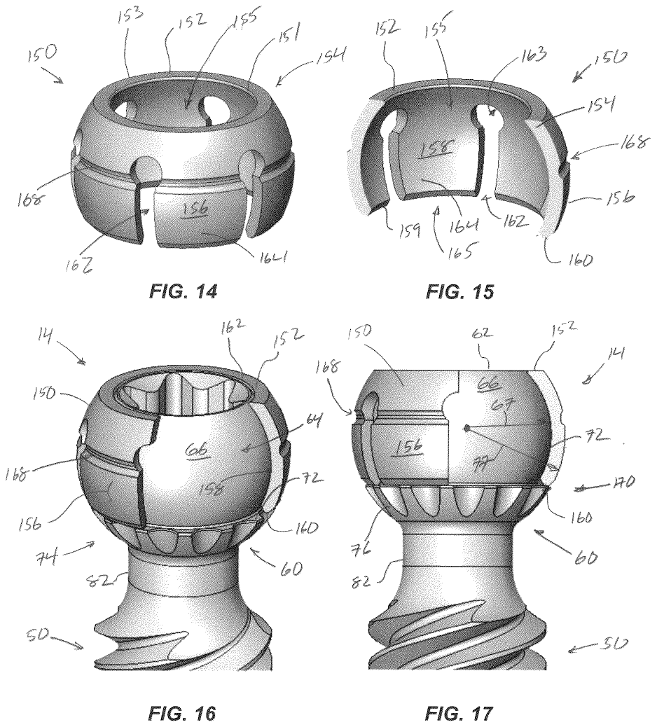

Additional discussion of the structure and features of the bone anchor 50 and its universal bi-spheric capture portion 60 is provided in co-owned U.S. Pat. No. 12,414,801, filed Nov. 3, 2023, which is incorporated by reference in its entirety herein and for all purposes. Accordingly, a detailed discussion of the additional structures and features of the bone anchor 50 and its bi-spheric shank head 60, as well as the bi-spheric shank head's connection with a cap retainer (such as the cap retainer 150 shown in FIGS. 14-17) and additional interactions with other components of the different receiver sub-assemblies 12, 22 of the present disclosure will not be repeated herein.

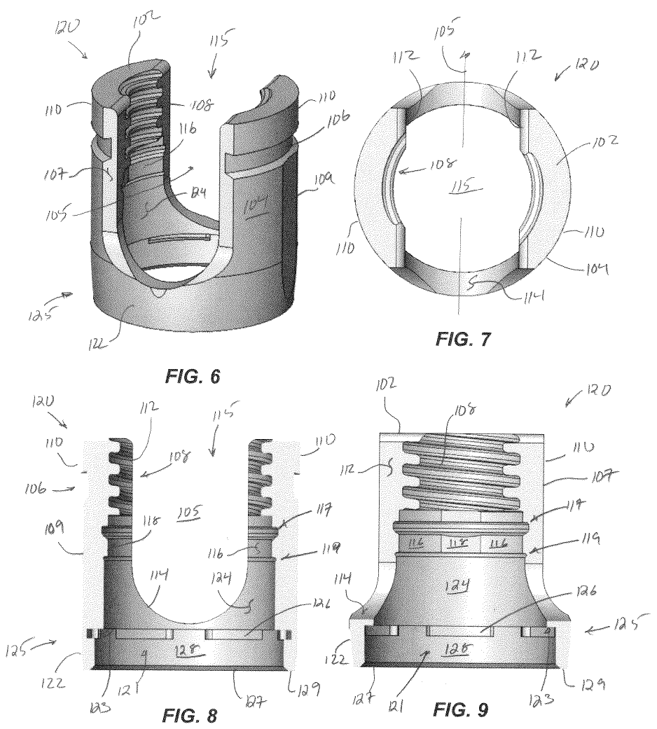

With reference now to FIGS. 6-13, the receiver 100 can be formed as a two-piece body comprising the circular base 140 that can be attached or coupled to the upper body 120 after the cap retainer 150 and the collet insert 170 had been pre-assembled into the upper body 120. It will be appreciated that both the upper body 120 and the circular base 140 can have substantially-cylindrical or circular shapes that can also include flat portions, grooves or recesses, flared portions, flanges, and apertures, etc., formed into outer surfaces, as well as an annular sub-structure (or first connection structure) extending downward at the lower end portion of the upper body and an annular super-structure (or second connection structure) extending upward from an upper end portion of the circular base 140.

Shown in FIGS. 6-9 is the upper body 120 of the two-piece receiver 100 that includes the open rod channel 105, as defined by the pair of upright arms 110, and the central bore 115 that extends upwardly from the annular lower end 125 through the rod channel 105 to the top surfaces 102 of the upright arms 110. The upper or channel portion of the central bore 115 further includes a discontinuous guide and advancement structure 108 formed into the interior faces of the upright arms 110, which guide and advancement structure 108 is configured to engage with a complementary structure formed into the outer side surfaces of the closure 190 (see FIGS. 31-32), as described more fully below. In one aspect the inner flange or crest surfaces of the discontinuous guide and advancement structure 108 can define the narrowest portion of the central bore 115, while vertical planar end surfaces 112 on either side of the guide and advancement structure 108 and saddle surfaces 114 can define the front and back ends of the rod channel 105 that are adjacent the front and back faces of the two-piece receiver 100.

In one aspect the guide and advancement structure 108 can be a discontinuous helically wound interlocking flange form. It will be understood, however, that the guide and advancement structure 108 could alternatively comprise a square-shaped thread, a buttress thread, a modified buttress thread, a reverse angle thread, or other thread-like or non-thread-like closure mating structure for operably guiding the closure downward between the upright arms 110 under rotation until the closure directly engages and presses against the elongate rod positioned within the channel 105. Additionally, the various structures and surfaces forming a helically wound guide and advancement structure 108 can also be configured to resist, to inhibit, to limit, or to preferentially allow and control some limited amount of splay of the upright arms 110 of the receiver 100 while advancing the closure downward under rotation and when torquing the closure against the elongate rod to generate a downwardly-directed thrust that locks the completely assembled multiplanar bone anchor assembly into position.

Moving downward along the interior faces of the upright arms 110, the upper portion of the central bore 115 located between the vertical end surfaces 112 that define the rod channel 105 can include a discontinuous upper cylindrical surface 116 immediately below the guide and advancement structure 108. The upper cylindrical surface 116 can have an inner diameter that is greater than the inner flange or crest diameter of the helically-wound interlocking flange form defining the guide and advancement structure 108. In addition, it is foreseen that a run-out groove or grooves may also be formed into the interior faces of the upright arms between the guide and advancement structure 108 and the upper cylindrical surface 116.

The internal surfaces of the central bore 115 of the upper body 120 can also include features that are complementary with the outer surfaces of the collet insert 170, so as to allow the collet insert 170 to be slidably received and/or secured therein and to be maintained in alignment with respect to the open rod channel 105. For instance, the discontinuous upper cylindrical surface 116 be bisected into upper and lower portions by opposed shipping state grooves 117. The shipping state grooves 117 can extend across the width of the interior faces of each upright arm 110 to the vertical end surfaces 112 that define the front and back ends of the channel 105. As described below, the shipping state grooves 117 can allow for upper corner ridges of the collet insert 170 to be uploaded into position within the shipping state grooves 117 to secure an initial vertical or axial position of the collet insert 170 within the central bore 115. In one aspect the discontinuous upper cylindrical surface 116 can be further divided by centered inner planar surfaces 118 that are configured to slidably receive complementary outer planar surfaces of the collet insert 170 so as to maintain the insert channel of the collet insert 170 in alignment with open rod channel 105, also described in more detail below.

As shown in the drawing figures, the upper surfaces of the opposed shipping state grooves 117 can be downward-facing arcuate planar surfaces configured to engage with planar upper surfaces of the upper corner ridges, so as to prevent the collet insert 170 from moving upward within the central bore 115 after the upper corner ridges have been uploaded into the shipping state grooves 117. In contrast, the lower surfaces of the shipping state grooves 117 can comprise ramped surfaces that extend downwardly and inwardly toward the lower portion of upper cylindrical surface 116 located just below the shipping state grooves 117.

Moving downward through the central bore 115, a discontinuous center cylindrical surface 124 can be located below the lower portion of the discontinuous upper cylindrical surface 116, with opposed lower locking grooves 119 being formed into the interior surfaces of the upright arms 110 at the junction between the two discontinuous cylindrical surfaces 116, 124. The discontinuous center cylindrical surface 124 can have an internal diameter that is greater than the internal diameter of the discontinuous upper cylindrical surface 116, such that upper surfaces of the lower locking grooves 119 can be downward-facing arcuate planar surfaces configured to engage with the same planar upper surfaces of the upper corner ridges of the collet insert 170 after the collet insert 170 has been pushed downwardly across the axial width of the lower portion of the discontinuous upper cylindrical surface 116, as described below.

With continued reference to FIGS. 6-9, the annular lower end or sub-structure 125 of the upper body 120 (or first connection structure) can include a downwardly-extending annular skirt 122 with a circular lower edge surface 129 that defines a lower opening 121, and an interior lower cylindrical surface 128 extending downwardly from a downward-facing annular step surface 123 that defines the boundary between the lower cylindrical surface 128 and the discontinuous center cylindrical surface 124. The lower cylindrical surface 128 can have an inner diameter that is greater than the diameter of the discontinuous center cylindrical surface 124. In addition, a plurality of arcuate recessed cutouts 126 can be formed into the lower cylindrical surface 128 immediately below the annular step surface 123, with the recessed cutouts 126 being equally spaced around the circumference of the lower cylindrical surface 128.

The upper body 120 of the two-piece receiver can have a partially cylindrical and partially faceted outer profile. In the illustrated embodiment, for example, the partially cylindrical portions can include curvate side outer surfaces 104 of the upright arms 110 that extend downward from the top surfaces 102 toward the annular lower end 125 of the upper body 120. The upper body 120 can further include upper curvate-extending instrument engaging grooves 106 below the top surfaces 102 of the upright arms 110 that extend horizontally across the curvate side outer surfaces 104, and in one aspect can extend to the front face and the back face of the two-piece receiver.

As shown in the drawings, the outer surfaces of the upper body 120 can also include front and back narrow flats 107 or tool engagement features on the front and back faces of the upright arms 110, as well as side outer planar faces 109 and/or tool receiving and engaging side recesses (not shown) formed into the curvate side outer surfaces 104 below the upper instrument engaging grooves 106, and which can be parallel with each other and oriented perpendicular to the front and back narrow flats 107. In one aspect the upper instrument engaging grooves 106, the narrow flats 107, the side outer planar faces 109, and any other planar tool-engagement surface or recess can serve together as outer tool engagement surfaces that allow for tooling to more securely engage and hold the upper body 120 during an initial pre-assembly with the internal components to form the multiplanar receiver sub-assembly 12, during coupling of the receiver sub-assembly to the bone anchor 50, either after or before the implantation of the anchor portion 84 of the bone anchor into a vertebra, and also during further assembly of the multiplanar receiver sub-assembly 20 with the elongate rod and the closure so as to aid in torquing and counter-torquing to lock the assembly.

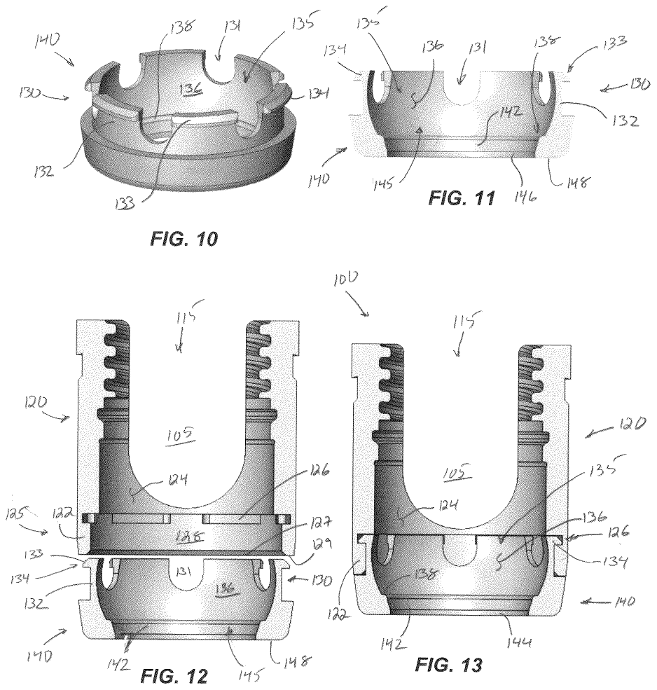

With reference to FIGS. 10-11, the circular base 140 can define the internal cavity 135 of the two-piece receiver 100 that communicates with the bottom surface 148 of the circular base 140 through a bottom opening 145. The lower portion of the internal cavity 135 can include a spherical seating surface 142 that is sized and shaped to closely receive the spherical outer surface of the cap retainer 150. The spherical seating surface 142 can extend to an upward-facing annular shelf surface 138 that defines the upper border of the seating surface 142. In one aspect a bottom cylindrical surface 146 can extend downwardly from the lower edge 144 of the spherical seating surface 142 to the bottom surface 148 of the circular base 140 to define the bottom opening 145.

The circular base 140 can also include an annular upper or super structure 130 (or second connection structure) that is configured to engage with the annular lower end 125 of the upper body 120 to secure the upper body 120 to the circular base 140. The annular super structure 130 can include opposing sets of upwardly-extending flexible panels or spring tabs 132, separated by slots 131, and having curvate inner surfaces 136 that can define an upper expansion portion of the internal cavity 135. The spring tabs 132 can further includes outwardly-projecting protuberances or flanges 134 at their upper ends. The outwardly-projecting flanges 134 can be sized and shaped to enter into the arcuate recessed cutouts 126 formed into the lower inner cylindrical surface 228 adjacent the annular step surface 123 (see FIGS. 6-9) so that the two pieces can engage in a configuration that prevents rotation between the upper body 220 and the cylindrical base 240. In one aspect the outwardly-projecting flanges 134 can include beveled top surfaces 133 configured to engage with the tapered inner surface 127 extending between the lower inner cylindrical surface 228 and the annular lower edge surface 129 of the upper body.

With reference to FIGS. 12-13, in one aspect the means or mechanism for coupling together the upper body 120 and the circular base 140 to form the two-piece receiver 100 can comprise a type of spring-latch connection. For example, an initial engagement between the beveled top surfaces 133 of the spring tabs 132 and the tapered inner surface 127 of the annular skirt 122 can cause the spring tabs 132 to flex inward so that the outer tip surfaces of the outwardly-projecting flanges 134 ride upwards along the lower inner cylindrical surface 128 of the annular skirt 122. The spring tabs 132 can continue to move upward until the outwardly-projecting flanges 134 reach the level of the recessed cutouts 126, at which point the flanges 134 of the plurality of spring tabs 132 can snap or latch into the circular formation of the plurality of recessed cutouts 126, thereby coupling together the upper body 120 and cylindrical base 140 of the two-piece receiver 100.

With reference to FIGS. 14-15, the multiplanar cap retainer 150 can have the form of a hollow, partial spherical shell with a solid or continuous upper ring portion 154 having an annular planar upper surface 152 with a continuous circular inner edge 151 that defines a central upper opening 155. As can be seen in the drawings, a discontinuous outer spherical surface 156 extends downward from the continuous circular outer edge 153 of the upper surface 152 toward a discontinuous annular bottom surface 160, and a discontinuous inner spherical surface 158 extends downward from the circular inner edge 151 of the upper surface 152 toward the discontinuous annular bottom surface 160. The distance between the discontinuous inner spherical surface 158 and the discontinuous outer spherical surface 156 can define the thickness of the partial spherical shell that forms the cap retainer. The cap retainer 150 can further include a discontinuous horizontal groove 168 formed into and extending circumferentially around the discontinuous outer spherical surface 156 of the cap retainer 150, at about the midline or equator of the discontinuous outer spherical surface 156.

A plurality of slots 162 can be formed through the thickness of the cap retainer 150, from the discontinuous inner spherical surface 158 to the discontinuous outer spherical surface 156 and extending upward from the discontinuous annular bottom surface 160 toward the continuous circular upper ring portion 154. The slots 162 can be equally spaced around the circumference of the cap retainer 150 to form a plurality of flexible collet fingers 164 extending downward from the upper ring portion, and which collet fingers 164 can flex outwardly at their lower ends, so as to expand a central lower opening 165 of the cap retainer 150 to receive the bi-spheric shank head 60 of the shank 50. It will be appreciated that the discontinuous outer spherical surface 156, the discontinuous inner spherical surface 158, and the discontinuous annular bottom surface 160 can be considered ‘discontinuous’ due to the interruptions in the surfaces created by plurality of slots extending upwardly through the lower edge and thickness of the lower portions of the shell forming the cap retainer 150, and that other terminology may also be applicable.

As shown in the drawings, the cap retainer 150 can also include an inner beveled edge surfaces 159 between the discontinuous bottom annular surface 160 and the discontinuous inner spherical surface 158, which inner beveled edge surfaces 159 can define the expandable central lower opening 165 of the cap retainer. In one aspect the upper ends of the slots 162 formed through the thickness of the cap retainer can also be formed as rounded apertures 163 or curved stress-relieving end passages.

The diameter of the discontinuous inner spherical surface 158 of the cap retainer 150 is substantially equal to the minor diameter 67 defined by the upper spherical surface 66 of the bi-spheric shank head 60 (see FIG. 4), while the diameter of the discontinuous outer spherical surface 156 is substantially equal to the major diameter 77 defined by both the lower spherical surface 76 of the bi-spheric shank head 60 and the spherical seating surface 142 of the two-piece receiver 100. As such, the cap retainer 150 can be sized and shaped so that once positioned on the bi-spheric shank head 60, as shown in FIGS. 16-17, the discontinuous inner spherical surface 158 can mate or engage with the upper spherical surface 66 while the discontinuous annular bottom surface 160 engages with the lower upward-facing shelf or ledge 72 of the bi-spheric shank head. In this coupled or captured configuration, in one aspect the annular planar upper surface 152 of the cap retainer 150 can be substantially flush or aligned with the annular planar top surface 62 of the bi-spheric shank head 60. In addition, the discontinuous outer spherical surface 156 of the cap retainer 150 can also be aligned with the lower spherical surface 76 of the lower partial spherical portion 74 so as to create a single diameter, articulating, multiplanar shank head sub-assembly 12 having the major diameter 77 that is greater than the diameter of the bottom opening 145 of the receiver 100, thereby preventing the bottom-loaded shank 50 from exiting the receiver 100 back out through the same bottom opening 145 through which it was initially loaded. It will be appreciated that the cap retainer 150 can still remain a member of the receiver sub-assembly 12 even after its coupling to the bi-spheric shank head 60 to form the shank head sub-assembly 14, and as such may be considered the linking mechanism that connects the two sub-assemblies together.

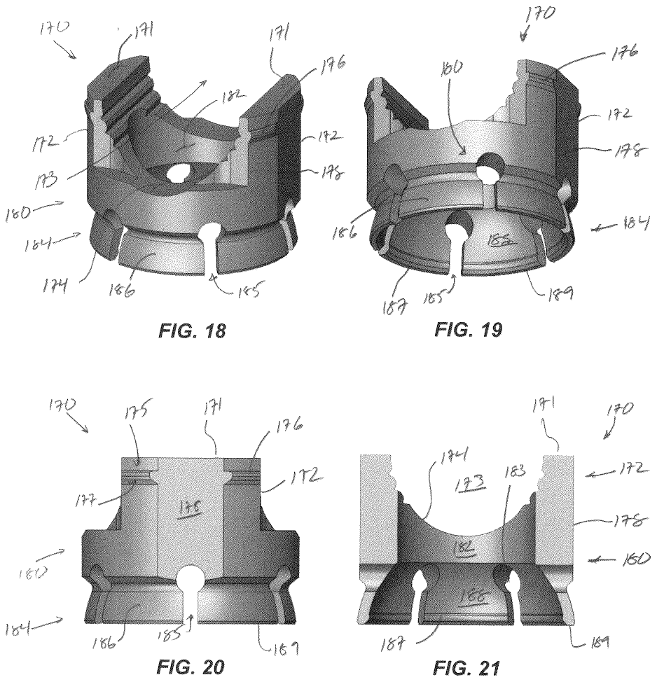

Illustrated in FIGS. 18-21 is the collet insert 170 that generally includes a circular center portion 180, a pair of insert arms 172 extending upward from the circular center portion 180 to define an insert channel 173, and a lower collet portion 184 extending downward from the circular center portion 180 to define a discontinuous, downwardly-opening concave inner spherical surface 188 that is engageable with the discontinuous spherical outer surface of the cap retainer. The collet insert 170 can have a generally-cylindrical shape that is sized to be slidably received within the central bore of the multiplanar receiver. The insert arms 172 can form the insert channel 173 extending therebetween that is alignable with the rod channel of the receiver after the collet insert 170 has been positioned within the central bore, and which insert channel 173 can be further defined by an upward-facing rod seating surface 174 extending between the insert arms 172 that is engageable with the cylindrical elongate rod. The collet insert 170 can further include a central tool-receiving aperture 183 defined by an inner cylindrical surface 182 that is configured to slidably receive a drive tool (not shown) that extends downwardly through the central bore of the multiplanar receiver to engage the internal drive socket formed into the top end of the bi-spheric shank head.

The lower collet portion 184 can comprise a discontinuous curvate skirt extending downwardly and outwardly from the circular center portion 180 toward a discontinuous bottom edge surface 189, and having plurality of slots 185 formed through the thickness of the curvate skirt, from the discontinuous outer spherical surface 187 to the discontinuous inner spherical surface 188 and extending upward from the discontinuous bottom edge surface 189 toward the continuous circular center portion 180. The slots 185 can be equally spaced around the circumference of the curvate skirt to form a plurality of flexible collet fingers 186 extending downward from the circular center portion 180, and which collet fingers 186 can flex outwardly at their lower ends, so as to expand a central lower opening of the collet insert 170 to receive the cap retainer 150. It will be appreciated that the discontinuous outer spherical surface 187, the discontinuous inner spherical surface 188, and the discontinuous bottom edge surface 189 can be considered ‘discontinuous’ due to the interruptions in the surfaces created by plurality of slots 185 extending upwardly through the lower edge 189 and extending laterally through the thickness of the curvate skirt forming the lower collet portion 184, and that other terminology may also be applicable.

The lower collet portion 184 can further include a discontinuous, inwardly-protruding lower ridge 187 at the bottom edges of the collet fingers 186 that is complementary with the horizontal groove 168 of the cap retainer 150, so as to enter the horizontal groove 168 upon the pre-assembly of the cap retainer 150 and the collet insert 170 into the two-piece receiver 100 to form the multiplanar receiver sub-assembly 12 in the shipping state configuration. In one aspect the circular tongue-and-groove type engagement between the discontinuous horizontal groove 168 of the cap retainer 150 and the discontinuous, inwardly-protruding lower ridge 187 of the collet portion 184 of the collet insert 170 can serve to better hold the cap retainer 150 in the stabilized and centralized position within the internal cavity 135 above the bottom opening 145, so as to prevent the cap retainer 150 from shifting or pivoting or otherwise allowing the center aperture of the cap retainer 150 to become mis-aligned relative to the bottom opening 145 of the receiver 100 in a way that would hinder or prevent the uploading of the bi-spheric shank head 60 into the multi-planar receiver sub-assembly 12.

As shown in the drawing figures, the insert arms 172 can include upper corner ridges 176 that project radially outward from the outer surfaces of the insert arms 172 that, in turn, can flex inward during the uploading of the collet insert 170 so as to allow the four upper corner ridges 176 to slide upwards into the upper shipping state grooves 117 formed into the central bore 115 as the collet insert 170 is uploaded into the upper body 120 of the two-piece receiver 100. The upper corner ridges 176 can include upward-facing arcuate planar upper surfaces 175 configured to abutingly engage the downward-facing arcuate planar surfaces of the opposed shipping state grooves 117, as well as downward-facing arcuate ramped or tapered lower surfaces 177 configured to slidably engage the ramped lower surfaces of the shipping state grooves 117.

The multiplanar collet insert 170 may also include an indexing structure configured to slidably engage with a complementary indexing structure formed into the central bore of the multiplanar receiver, upon the uploading of the collet insert 170 into the upper body 120 of the two-piece receiver 100, so as to inhibit rotation of the insert out of its initial shipping state position. For example, in one embodiment the indexing structure of the insert can comprise opposite planar side surfaces 178 formed into the outer surfaces of the collet insert 170. The planar side surfaces 178 can slidably engage the centered inner planar surfaces 118 formed into the central bore 115 of the upper body 120 during the uploading of the collet insert 170 into its shipping state position. It is foreseen that other structures can be used to hold the insert relative to the receiver, such as indexing nubs, crimps, pegs, set screws or separate rings, to inhibit rotational movement and/or to control translational movement of the insert along the vertical axis of the receiver, and that the collet insert 170 could be snapped in place, or otherwise positioned, within the upper body 120 of the two-piece receiver.

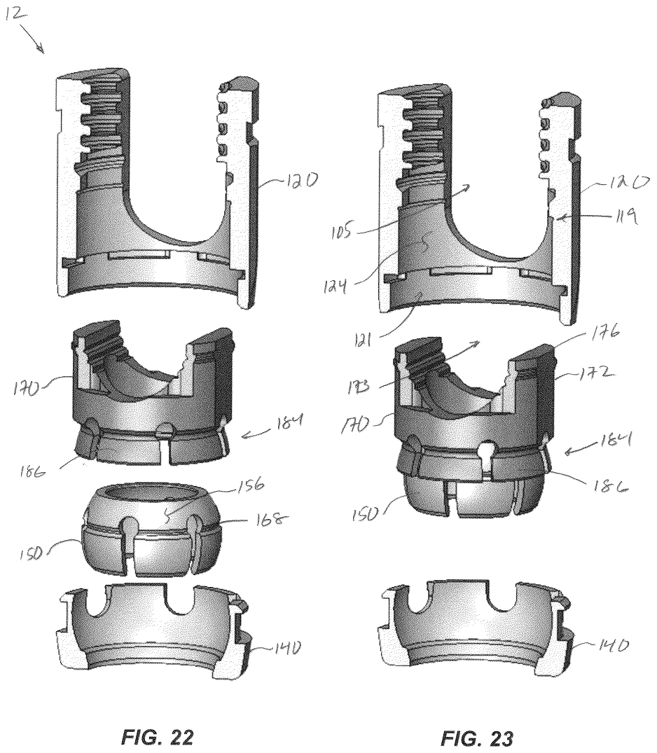

Illustrated in FIG. 22 are the individual components of the multiplanar pivotal bone anchor assembly 10 that, in many embodiments, can be pre-assembled together into a receiver sub-assembly 12 at a factory or manufacturing facility, prior to shipping to a spine company or a hospital or surgery center and engagement with the capture portion of the bone anchor in the surgical setting. As described above, these components generally include the two-piece receiver 100 comprising the cylindrical base 140 and upper body 120, the cap retainer 150, and the collet insert 170. In one aspect the two-piece receiver 100, the cap retainer 150, and the collet insert 170 being pre-assembled into a multiplanar receiver sub-assembly 12 can be further defined as the shipping state configuration for the ‘modular’ bone anchor assembly, as described herein and commonly understood in the art. It will be appreciated, however, that in other embodiments the shipping state configuration can include the additional assembly of the multiplanar receiver sub-assembly together with the bone anchor at the factory or manufacturing facility or the spine company. It will also be appreciated that in yet other embodiments the individual components described above can also be pre-assembled into the receiver sub-assembly at the hospital or surgery center prior to implantation in a patient.

To begin the pre-assembly of the receiver sub-assembly 12, that cap retainer 150 can first be uploaded in the lower collet portion 184 of the collet insert 170, with the curvate collet fingers 186 flexing open until the cap retainer 150 is captured by the lower collet portion 184. It will be appreciated that the cap retainer can be captured in a stabilized position in which the discontinuous outer spherical surface 156 of the cap retainer 150 is frictionally engaged with the discontinuous inner spherical surface 188 of the lower collet portion 184, and the discontinuous, inwardly-protruding lower ridge 187 at the bottom edges of the collet fingers 186 is received within the horizontal groove 168 of the cap retainer 150, as shown in FIG. 23.

After the cap retainer 150 is captured by the lower collet portion 184 of the collet insert 170, the collet insert 170 and cap retainer 150 may then be uploaded together into the central bore 115 of the upper body 120 and installed into its the shipping state position. As shown in FIG. 23, this can be achieved by first positioning the collet insert 170 and captured cap retainer 150 below the lower opening 121 of the upper body 120 with the insert channel 173 being aligned with the rod channel 105.

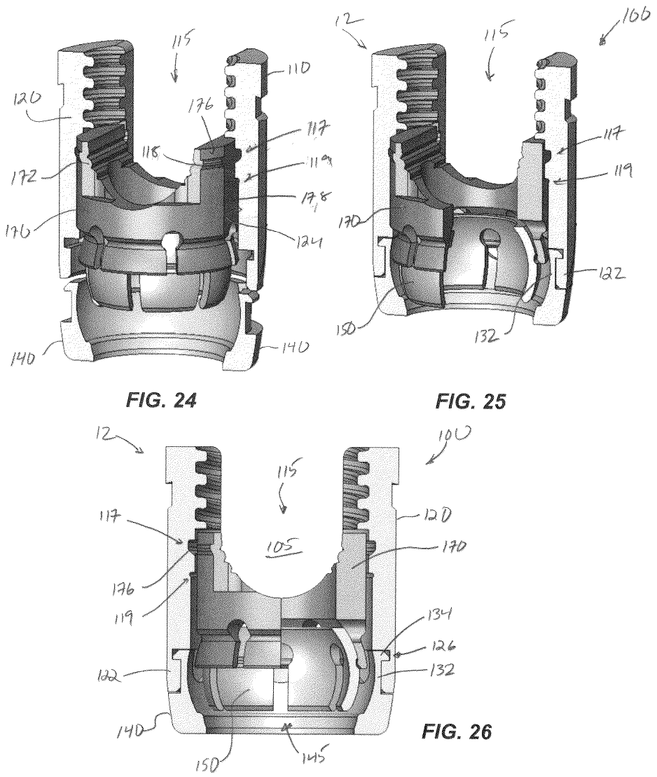

With reference to FIG. 24, the collet insert 170 and captured cap retainer 150 can then be uploaded into the central bore 115 through the lower opening 121, with the upper corner ridges 176 sliding upwardly along the discontinuous center cylindrical surface 124 until upper portions of the opposite planar side surfaces 178 slidably engage with the centered inner planar surfaces 118 of the central bore 115 and the upper corner ridges 176 reach the level of the downward-facing arcuate planar surfaces that form the upper surfaces of the opposed locking grooves 119. The insert arms 172 can then be deflected or flexed inwardly to allow the upper corner ridges 176 to move upwardly past the downward-facing arcuate planar surfaces of the opposed locking grooves 119 to reach the lower portions of the discontinuous upper cylindrical surface 116. The collet insert 170 and captured cap retainer 150 can then continue to move upward until the upper corner ridges 176 reach the level of and snap into the opposed shipping state grooves 117.

With reference to FIGS. 25-26, in one aspect the means or mechanism for coupling together the upper body 120 and the cylindrical base 140 can comprise the type of spring latch connection described above. In particular, the plurality of flexible curvate panels or spring tabs 132 having outwardly-projecting flanges or hook structures 134 at their upper ends can be formed into the annular upper structure 130 of the cylindrical base 140. The outwardly-projecting hook structures 134 can be sized and shaped to enter into the arcuate recessed cutouts 128 formed into the lower inner cylindrical surface 228 of the upper body 120. As described above, an initial engagement between the beveled upper edges 133 of the spring tabs 132 and the tapered inner surface 127 of the annular skirt 122 can cause the spring tabs 132 to flex inward so that the outer tip surfaces of the outwardly-projecting flanges 134 ride upwards along the lower inner cylindrical surface 128 of the annular skirt 122. The spring tabs 132 can continue to move upward until the outwardly-projecting flanges 134 reach the level of the arcuate recessed cutouts 126, at which point the flanges 134 of the plurality of spring tabs 132 can snap or latch into the circular formation of the plurality of recessed cutouts 126, thereby coupling together the upper body 120 and cylindrical base 140 to complete the pre-assembly of the two-piece receiver 100.

The coupling together of the upper body 120 and with cylindrical base 140 to form the two-piece receiver 100, as shown in the drawings, can comprise the final steps for pre-assembling the multiplanar receiver sub-assembly 12 into its shipping state position or configuration. It will be appreciated that the receiver sub-assembly 12 in its shipping state configuration is configured to prevent both the collet insert 170 and the cap retainer 150 from exiting the central bore 115 of the two-piece receiver 100 and/or from moving out of alignment. In other words, the pre-assembly together of the two-piece receiver 100, the cap retainer 150, and the collet insert 180 to form the multiplanar receiver sub-assembly 12 in the shipping state configuration, in which collet insert 170 is secured in an aligned position within the rod channel 105 and the cap retainer 150 is stabilized and centralized above the bottom opening 145, is now complete.

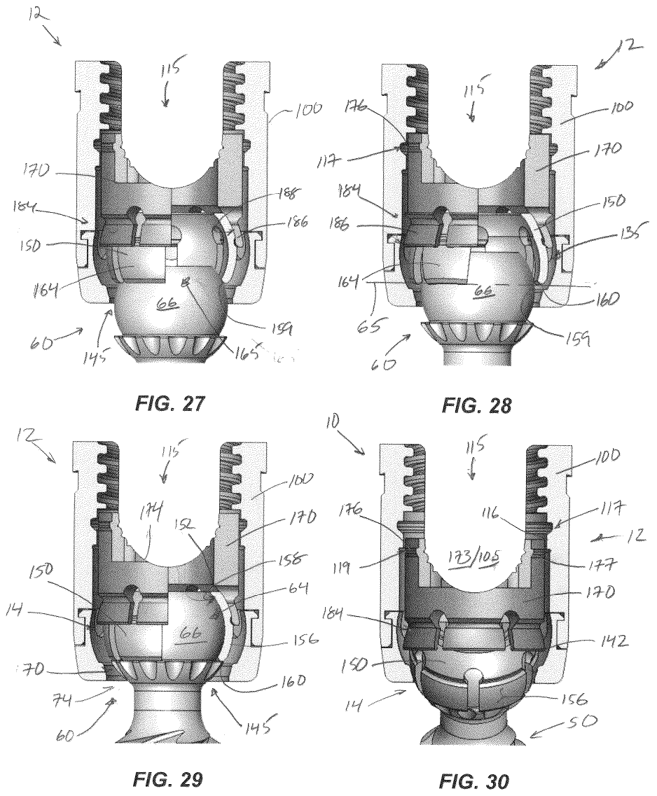

One representative embodiment or method of assembling the multiplanar receiver sub-assembly 12 to the bi-spheric shank head 60 of the bone anchor or shank 50 is illustrated in FIGS. 27-30. For instance, and with initial reference to FIG. 27, the receiver sub-assembly 12 can be first positioned above the proximal end of the bone anchor 50 with the expandable central lower opening 165 of the cap retainer 150, which is stabilized and centered above the bottom opening 145 of the receiver 100 by the lower collet portion 184 of the collet insert 170, being generally aligned with the upper partial spherical portion 64 of the bi-spheric shank head 60. The receiver sub-assembly 12 is then dropped downward (or the bone anchor is moved upward, depending on the frame of reference of the reader) until the upper spherical surface 66 of the bi-spheric shank head 60 passes upward through the bottom opening 145 of the receiver 100 to reaches and begin to push against the beveled edge surfaces 159 of the collet fingers 164 of the cap retainer 150.

As shown in FIG. 28, the receiver sub-assembly 12 can continue to move downward (or the bone anchor moves upward) as the bi-spheric shank head 60 begins to push against the beveled edge surfaces 159 of the collet fingers 164 of the cap retainer 150 that is held in space within the internal cavity 135 by the lower collet portion 184 of the collet insert 170. The collet insert 170, in turn, is itself is upwardly immovable due to its engagement with the receiver 100 (via the upper outer ridges 176 being positioned within the upper shipping state grooves 117). Due to this series of direct rigid engagements, the cap retainer 150 does not move upward and instead the central lower opening 165 of the cap retainer 150 can expand as both the collet fingers 164 of the cap retainer 150 and the collet fingers 186 of the collet insert 170 are pushed apart by the upwardly-moving bi-spheric shank head 60. The collet fingers 164 of the cap retainer 150 and the collet fingers 186 of the collet insert 170 can continue to expand within the internal cavity 135 by the upward movement of the bi-spheric shank head 60 into the receiver 100, until the discontinuous annular bottom surface 160 of the cap retainer 150 reaches the level of the hemisphere plane 65 of the bi-spheric shank head 60 and the collet fingers 164 of the cap retainer 150 are at their point of maximum expansion.

With reference to FIG. 29, the receiver sub-assembly 12 can continue to move downward (or the bone anchor moves upward) until the upper partial spherical portion 64 of the bi-spheric shank head 60 becomes fully captured by the cap retainer 150 as it contracts to close around the upper partial spherical portion 64, so that the discontinuous inner spherical surface 158 of the cap retainer 150 is now secured around the upper spherical surface 66 of the bi-spheric shank head 60. Furthermore, with the simultaneous engagement of the discontinuous bottom annular surface 160 against the lower ledge 70 of the lower partial spherical portion 74, the cap retainer 150 is also now aligned on the bi-spheric shank head 60 so that the annular planar upper surface 152 that defines the central upper opening of the cap retainer 150 can be centered about the internal drive feature or drive socket of the bi-spheric shank head 60. In addition, the discontinuous outer spherical surface 156 of the cap retainer 150 can also be aligned with the lower spherical surface 76 of the lower partial spherical portion 74 so as to create the single diameter, articulating, multiplanar shank head sub-assembly 14 having the major diameter 77 (see FIG. 17) that is greater than the diameter of the bottom opening 145 of the receiver 100, thereby preventing the bottom loaded shank 50 from exiting the receiver 100 back out through the same bottom opening 145 through which it was initially loaded. It will be appreciated that the cap retainer 150 can still remain a member of the receiver sub-assembly 12 even after its coupling to the bi-spheric shank head 60 to form the shank head sub-assembly 14, and as such may be considered the linking mechanism that connects the two sub-assemblies together.

With the shank head sub-assembly 14 secured within the lower collet portion 184, the collet insert 170 can then be downwardly deployed with a deployment tool (not shown). In one aspect the deployment tool can include a rounded lower surface that is complementary with the upward-facing rod seating surface 174 of the insert channel 173 of the collet insert 170. However, it is foreseen that a variety of other structural features for providing contact engagement between the deployment tool and the collet insert 170 are also possible and considered to fall within the scope of the present disclosure.

With reference to FIG. 30, the deployment tool can be used to drive the collet insert 170 downward within the central bore 115, which can push the ramped lower surfaces 177 of the upper outer ridges 176 downward along the ramped lower surfaces of the upper shipping state grooves 117 until the upper outer ridges 176 reach and scrape across the lower portion of the upper cylindrical surface 116, after which the upper outer ridges 176 snap under the upper arcuate planar surfaces of the lower locking recess 119. With the same motion the lower collet portion 184 and the captured shank head sub-assembly 14 are also driven downward until the lower portion of the discontinuous outer spherical surface 156 of the cap retainer 150 becomes engaged within the spherical seating surface 142 of the receiver 100.

It will be appreciated that the timing of the engagements between the upper outer ridges 176 and the upper arcuate planar surfaces of the lower locking recess 119, and between the discontinuous outer spherical surface 156 and the spherical seating surface 142, can be substantially simultaneous. Upon completion of the deployment and removal of the deployment tool, as shown in FIG. 30, the upper and lower engagements can serve to secure the collet insert 170 and the cap retainer 150 to the internal structures of the central bore 115, and thereafter prevent these components of the receiver sub-assembly 12 from moving back up (or down) within the two-piece receiver 100. In addition, collet insert can include an outer cylindrical surface that is configured for slidable engagement with upper portions of the curvate inner surfaces 136 of the flexible spring tabs 132 to prevent the outwardly-projecting flanges 134 of the flexible spring tabs from disengaging from the arcuate recessed cutouts 126 of the upper body 120.

The coupling of the universal bi-spheric shank head 60 of the bone anchor or shank 50 with the multiplanar receiver sub-assembly 12 can complete the formation of the multiplanar bone anchor assembly 10 in its initial configuration, one in which the multiplanar bone anchor assembly 10 is ready to be implanted into the vertebrae of a patient or to receive the elongate rod and the closure.

FIG. 30 provides partially-sectioned view of the multiplanar bone anchor assembly 10 upon the initial assembly of the multiplanar receiver sub-assembly 12 to the bi-spheric shank head 60, but prior to final assembly with the elongate rod and the closure top. In one aspect the articulating multiplanar shank head sub-assembly 14 (i.e. the multiplanar cap retainer 150 and the bi-spheric shank head 60) can be secured against the downwardly-opening concave inner spherical surface 188 of the collet insert 170 and the spherical seating surface 142 of the multiplanar receiver 100 with a non-floppy frictional engagement, or pre-lock friction fit, that allows for the bone anchor or shank 50 to both pivot and rotate relative to the receiver 100 as the outer surfaces of the shank head sub-assembly 14 (i.e., the discontinuous outer spherical surface 156 of the cap retainer 150 and the lower spherical surface 76 of the bi-spheric shank head 60) slidably frictionally engage, with some resistance, with the concave inner spherical surface 188 and the spherical seating surface 142 with a ball and socket-type connection. It will be appreciated that this friction fit can be provided by the engagement between the upper outer ridges 176 of the collet inset 170 and the upper arcuate planar surfaces of the lower locking recess 119 which, in turn, can be configured to provide a downwardly directed force to upper portions of the discontinuous outer spherical surface 156 of the cap retainer 150. This can create the initial non-floppy frictional engagements between the cap retainer 150, the lower collet portion 184 of the collet insert 170, and the spherical seating surface 142 of the two-piece receiver 100 that allows for movement of the shank head sub-assembly 14 relative to the receiver 100 with some resistance.

Illustrated in FIGS. 31-32 is the multiplanar bone anchor assembly 10 after final assembly with the elongate rod 4 and the single piece closure 190. In this configuration the collet insert 170 can be pressed further downward within the receiver 100 by the elongate rod 4 and closure 190 so as to increase the downwardly directed force applied to the shank head sub-assembly 14. In one aspect the lower locking recesses 119 can include ramped lower surfaces (not numbered) that are smaller than the ramped lower surfaces of the upper shipping state grooves 117, thereby allowing the collet insert 170 to be easily pressed downward within the central bore until a hard or full frictional lock between shank head sub-assembly 14, the lower collet portion 184 of the collet insert 170, and the spherical seating surface 142 of the two-piece receiver 100 is achieved, preventing further movement between the shank head sub-assembly 14 and the receiver 100.

Finally, it will be appreciated that subsequent limited unthreading or backing-off of the single piece closure 190 from the receiver 100, without removing the elongate rod 4 or completely detaching the closure 190, can remove the additional downwardly directed force that was provided by the closure 190, thereby releasing the hard lock and re-establishing the non-floppy, friction fit configuration between the components of the multiplanar bone anchor assembly 10. A slight wiggling of the multiplanar receiver sub-assembly 12 can then serve to re-mobilize the receiver sub-assembly 12 relative to the bi-spheric shank head 60 and allow its position to be adjusted prior to re-locking the multiplanar bone anchor assembly 10 in a new position with a hard lock using the closure 190.

Through continuous research and development of new bone anchor designs, it has been determined that the significant pressures or loads being transferred through the various components of the multiplanar bone anchor assembly 10 can be high enough to approach or even exceed the local yield strength of the metal material(s) that form the individual components. In one aspect these slight local inelastic deformations may be useful by causing the separate internal components of the bone anchor assembly to compress and bind together to form a more solidly locked assembly or unit upon final locking with the elongate rod and closure. Nevertheless, in most situations it is necessary to maintain a comfortable margin between the pressure loads carried by the component and the yield strength of the material in order to account for variations in tolerances and occasional inconsistencies in imperfect manufacturing processes, thereby ensuring that the components and assemblies do not fail during implantation and use and are reliably strong over time.

One complicating factor in maintaining these load-bearing margins in the different components is the desire to scale downward the designs and functionalities of adult-sized bone anchor assemblies that are generally sized for use with elongate rods or longitudinal connecting members having rod diameters of about 6.50 mm to about 5.50 mm or 5.0 mm. In particular, it may be desirable to scale the adult-sized designs downward so as to be used with small stature/pediatric or cervical applications having rod diameters of 5.0 to about 2.5 mm. With the smaller rod sizes, it is preferrable that the overall size of the bone anchor assembly, and in particular the size of the central bore and bottom opening of the receiver and the outer diameter of the closure, are also reduced. However, it is notable that while the internal components can be reduced in size to fit within the smaller receiver, these reductions can also result in additional limitations with assembling both the internal components of the receiver sub-assembly and the capture portion of the bone anchor into the smaller receiver.

One design factor that can remain substantially constant is the pressure load provided by the closure that is required to lock a pivotal bone anchor assembly, both large and small, into its final locked configuration. In designs where the pressure load remains substantially constant while the size of the components in the load path(s) supporting these pressure loads are reduced, the local reduction in cross-sectional area can quickly result in increased stress levels that reach or exceed the yield strength of the material. Consequently, the different internal components are often simplified and/or removed altogether in bone anchor assemblies designed for small stature/pediatric or cervical applications, which can also strip away the improved functionalities provided by those components in adult-sized bone anchor assemblies.

Through continued modeling, analysis, and testing, it has been discovered that one solution for overcoming these challenges is to separate a solid integral receiver into a two-piece receiver, as described above. Once separated, the upper portion 120 of the two-piece receiver 100 that defines the channel 105 and central bore 115 can be modified to receive a reduced-size elongate rod, closure, and at least the upper portion of the collet insert 170 that also receives the reduced-sized elongate rod. At the same time, the size of the cylindrical base 140 or lower portion of the two-piece receiver 100 that defines the internal cavity 135 and bottom opening 145 can be generally maintained to allow the lower ‘retaining’ components of the bone anchor assembly 10, that are defined by or received within the internal cavity, to preserve their adult-or near adult-sized dimensions, together with their larger cross-sectional areas and acceptable load-bearing margins.

As noted above, this novel approach to re-designing the receiver can also facilitate the pre-assembly of the separate internal components into the two-piece receiver 100 to form the receiver sub-assembly 12, with the internal components (e.g., the collet insert 170 and the cap retainer 150) being loadable into the upper or lower pieces of the two-piece receiver prior to their coupling together, as described above, rather than being downloaded through the central bore or uploaded through the bottom opening. It will be appreciated that with smaller-sized bone anchor assemblies, this approach can also preserve the use of the larger, higher-functional internal components that are configured to interface with the universal shank heads described above, so as to provide the bone anchor assembly or spinal fixation system with the same modular capabilities as their adult-sized counterparts. In other words, in addition to maintaining their greater load-bearing capabilities, the smaller-sized two-piece receiver sub-assemblies can also preserve the higher-level functionalities, including but not limited to multiplanar, monoplanar, monoaxial, favored-angle, and independent lock capabilities, etc., that are configured for coupling with an uploadable universal shank head, as provided by their comparable adult-sized bone anchor assemblies and spinal fixation systems.

In addition to the above, it will be appreciated the bone anchor assembly or spinal fixation system described above may be considered “modular” in the sense that any particular type of receiver sub-assembly, in the shipping state condition, can be coupled with any one of a variety of bone anchors 50 having anchor portions of different size, length, type, and/or thread patterns, but with all of the bone anchors 50 having the same universal capture structure, such as the bi-spheric shank head 60, at their upper ends. Furthermore, it will also be appreciated that a receiver sub-assembly 12 in the shipping state condition can be assembled with the bone anchor 50 at the hospital or surgery center either before insertion into the patient, or after the threaded shank or bone anchor 50 has been inserted into the patient (such as directly by a surgeon or with robotic assistance). The different techniques or approaches for the insertion and assembly of the modular parts of the bone anchor assembly can be described as ex-vivo and in-vivo or in-situ, respectively.

### Turret Bone Anchor Assembly

FIG. 33 is an exploded perspective view of another representative embodiment 20 of the multiplanar bone anchor assembly that is configured to provide a rotational turret-type functionality between the upper body 220 and circular base 240 of the two-piece receiver 200. In one aspect turret-type multiplanar (i.e., turret) bone anchor assembly 20 can include the same bone anchor or shank 50 described above, having a bi-spheric shank head 60 and an anchor portion 84 opposite the bi-spheric shank head 60 for securement or attachment to the bone of a patient. Alternatively, the turret bone anchor assembly 20 can include a smaller-sized bone anchor or shank 40 having a bi-spheric shank head 44 and an anchor portion 48 with a smaller diameters. Other features and aspects of the smaller-sized bone anchor 40 can be the same as or substantially similar to those of the larger-sized bone anchor or shank 50 described above.

As with the previous multiplanar embodiment, the turret bone anchor assembly 20 includes a turret-type two-piece receiver 200 or housing having a circular base 240 defining the lower portion of an internal cavity 235 that is configured to receive the capture portion 60 of the bone anchor 50, and an upper body 220 that includes a pair of upright arms 210 integrally formed with and extending upward from an annular lower end 225 to define an open rod channel 205 configured to receive an elongate rod 6 that can be smaller (i.e., with a smaller diameter) than the elongate rod described above. The annular lower end 225 and upright arms 210 can together define a central bore 215 that extends upward from the internal cavity 235 through the rod channel 205 to the tops of the upright arms 210, and which is centered about the vertical centerline axis 201 of the two-piece receiver 200.

The turret-type two-piece receiver 200 can be initially pivotably secured to the capture portion 44 of the bone anchor 40 with a number of separate internal components that have been pre-assembled into the internal cavity 235 and the central bore 215 to form a turret-type receiver sub-assembly 22. These internal components can include, but are not limited to, a pivoting or articulating multiplanar cap retainer 250 that is ultimately positioned in the internal cavity 235 or lower portion of the central bore 215, and which attaches to the bi-spheric shank head 44 to pivotably couple the shank 40 to the receiver sub-assembly 22. The receiver sub-assembly 22 further includes a collet insert 270 that can be positioned above the cap retainer 250 in a middle portion of the central bore 215 where the central bore 215 intersects with the rod channel 206, and is operable to engage with the cap retainer 250 below and to be engaged by the elongate rod 6 from above. In one aspect the collet insert 270 can be downwardly-displaceable with a tool or tooling from an upper shipping-state position to a lower friction-fit position after the bi-spheric shank head 44 has been uploaded into the receiver sub-assembly, so as to establish a non-floppy pre-lock friction fit configuration prior to final assembly with the elongate rod and the closure.

After the elongate rod 6 has been positioned within a lower portion of the rod channel and into engagement with the collet insert 270, a closure 290 can be threadably or otherwise secured into an upper portion of the central bore or rod channel to apply pressure to an upper surface of the rod 6, such as by direct contact, thereby locking both the elongate rod 6 and the turret bone anchor assembly 20 into a final locked configuration or position, such as that shown in FIGS. 51-52. It is foreseen that the collet insert 270 can also be downwardly-displaceable from the shipping state position to the friction fit and/or locked position simultaneous with the placement of the rod and threaded installation of the closure.

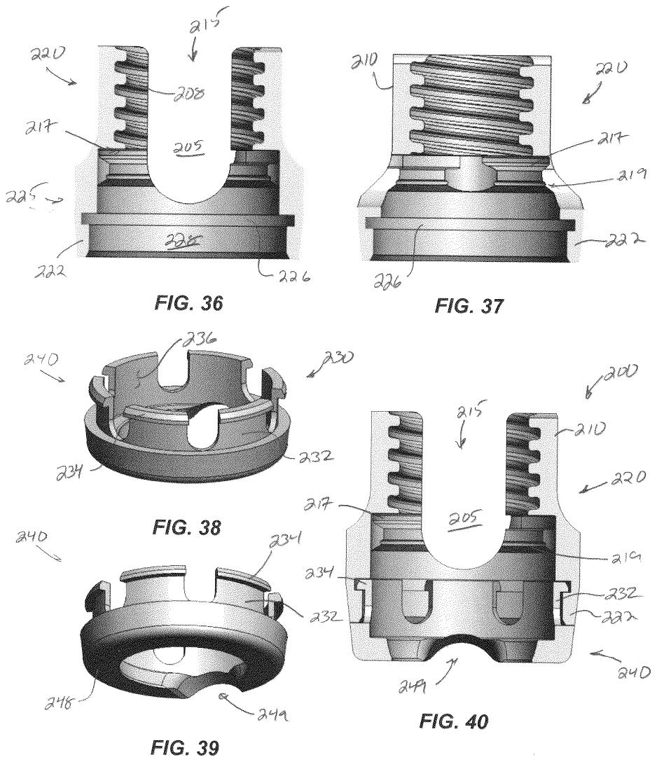

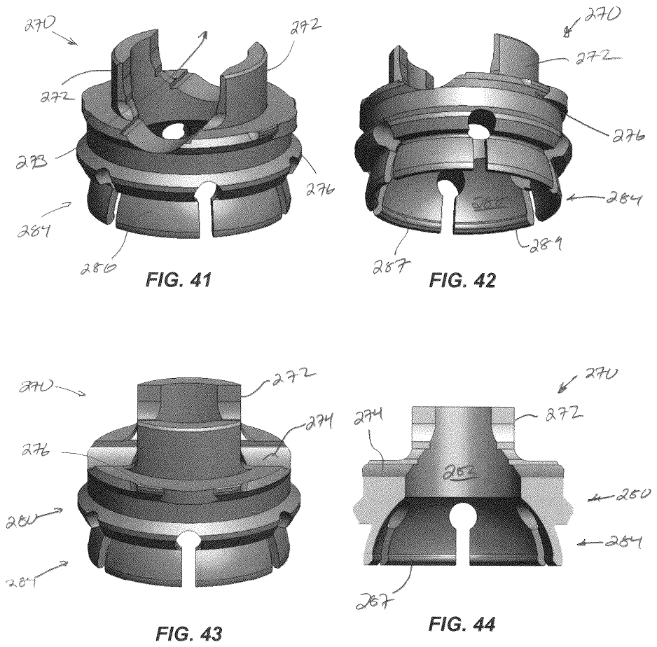

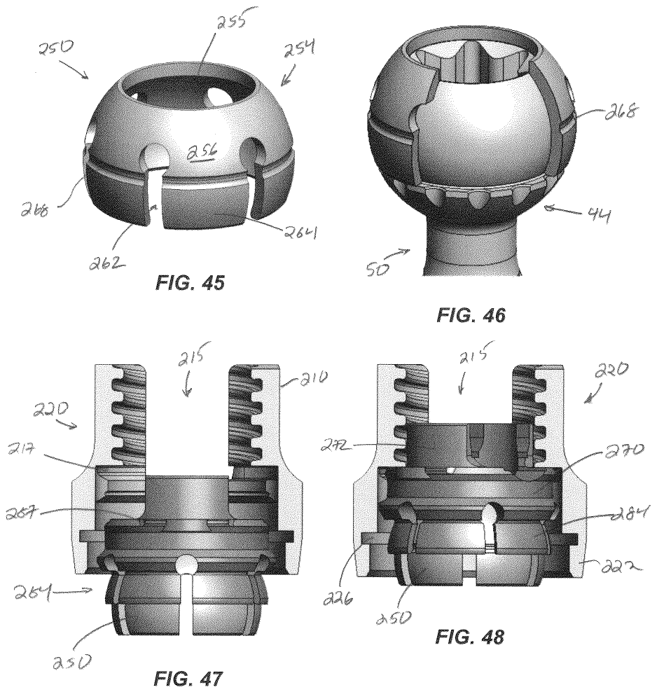

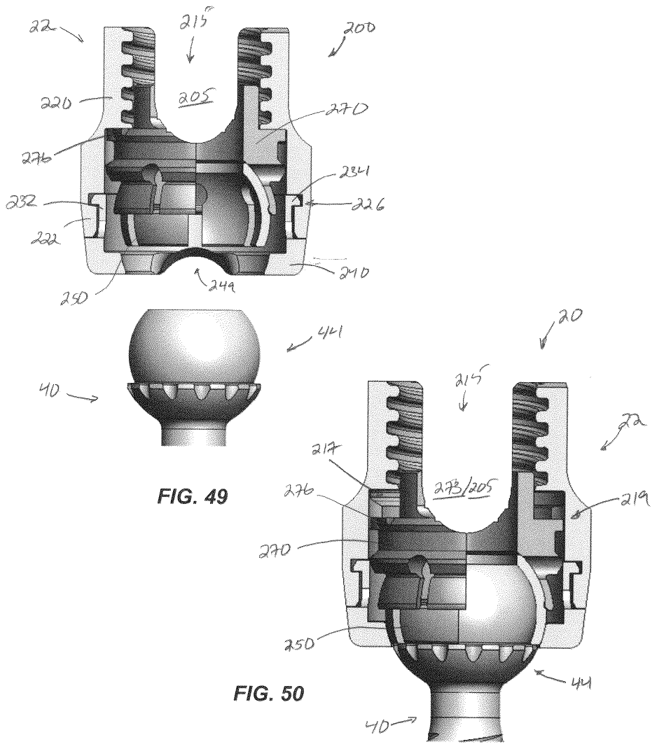

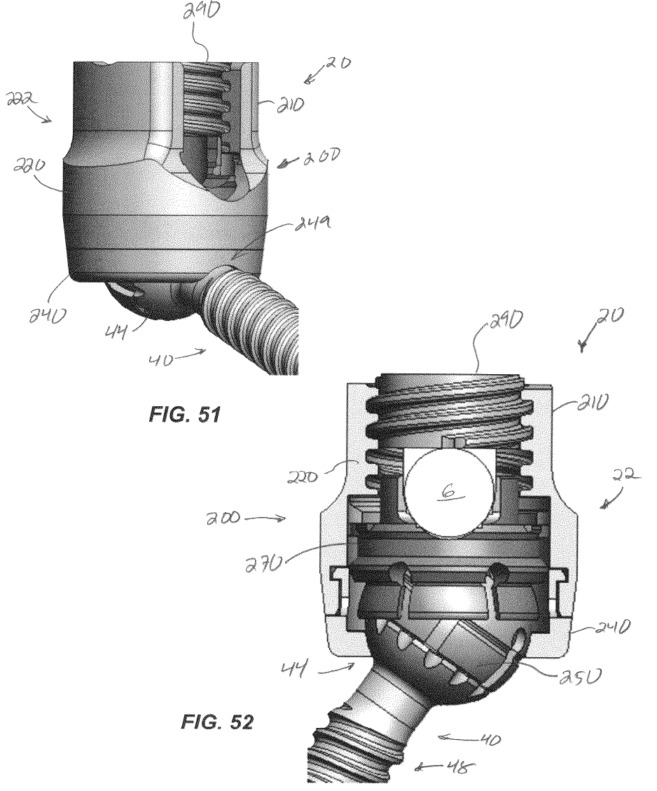

Differences between turret bone anchor assembly 20 of FIG. 33 and the multiplanar bone anchor assembly described above can include the replacement of the two-piece multiplanar receiver with a two-piece turret receiver 200, as shown in FIGS. 34-40. The two-piece turret receiver 200 can have many of the same features as the multiplanar version, with the exception that the arcuate recessed cutouts formed into the lower cylindrical surface of the two-piece multiplanar receiver can be replaced with a continuous circumferential recess 226 that allows for the outwardly-projecting protuberances or flanges 234 of the flexible spring tabs 232 to slide within the circumferential recess 226, thereby providing for turret-type rotation between the upper body 220 and circular base 240 of the two-piece turret receiver 200. In addition, a downward-facing semi-circular cut-out 249 can be formed into the bottom surface 248 of the circular base 240 to provide for increased pivotal angulation of the bone anchor or shank 40 relative to the two-piece turret receiver 200 in a single direction. In embodiments where the rod 6 comprises a smaller rod size, the upright arms 110 of the upper body 220 of the two-piece turret receiver 200 can also be reduced in size and moved closer together to define a narrower rod channel 205.

With reference to FIGS. 41-44, additional differences between the multiplanar bone anchor assembly and the turret bone anchor assembly 20 of FIG. 33 can also include the replacement of the multiplanar version of the collet insert with a turret collet insert 270. The turret collet insert 270 can have many of the same features as the multiplanar version, but with changes to the overall size and shape of the collet insert 270 that can result in the insert arms 272 being reduced in size and moved closer together to define a narrower insert channel 273.

With reference to FIGS. 45-46, additional differences between the multiplanar bone anchor assembly and the turret bone anchor assembly 20 of FIG. 33 can further include the replacement of the multiplanar version of the cap retainer with a turret cap retainer 250. The turret cap retainer 250 can include many of the same features as the multiplanar version, but with changes to the overall size and shape of the collet insert 270 in order to engage with the smaller-sized bi-spheric shank head 44 described above.

The pre-assembly of the turret cap retainer 250 and turret collet insert 270 with the two-piece turret receiver 200 to form the turret receiver sub-assembly 22 in the shipping state configuration can be substantially similar to the pre-assembly method for the multiplanar embodiment described above, with the completed turret receiver sub-assembly 22 shown in FIG. 49. Furthermore, the assembly of the turret receiver sub-assembly 22 with the bi-spheric shank head 44 can also be substantially similar to the assembly method for the multiplanar embodiment described above, with the complete the formation of the turret bone anchor assembly 20 shown in FIG. 50.

Illustrated in FIGS. 51-52 is the turret bone anchor assembly 20 after final assembly with the elongate rod 6 and the single piece closure 290. In this configuration turret bone anchor assembly 20 can include the same features and capabilities as described above with the multiplanar embodiment, with the addition that the circular base 240 can be rotatable about the vertical centerline axis 201 relative to the upper body 220. It will be appreciated that this capability allows for the direction of the increased pivotal angulation (or favored angle direction) of the bone anchor or shank 40 relative to the two-piece turret receiver 200 to be rotated 360 degrees to any angular relationship relative the elongate rod 6.

As indicated above, the invention has been described herein in terms of preferred embodiments and methodologies considered by the inventor to represent the best mode of carrying out the invention. It will be understood by the skilled artisan, however, that a wide range of additions, deletions, and modifications, both subtle and gross, may be made to the illustrated representative embodiments of the multiplanar bone anchor assembly without departing from the spirit and scope of the invention. As such, these and other revisions might be made by those of skill in the art without departing from the spirit and scope of the invention that is constrained only by the following claims.

What is claimed is:

1. A pivotal bone anchor assembly for securing an elongate rod to a bone of a patient with a closure, the pivotal bone anchor assembly comprising:
a two-piece receiver comprising:
an upper body comprising an annular lower end, a pair of upright arms extending upward from the annular lower end to define an open channel configured to receive the elongate rod, and a central bore centered about a vertical centerline axis and extending downward from tops of the upright arms through the open channel to the annular lower end, the annular lower end including a first connection structure extending downwardly opposite the pair of upright arms; and
a circular base defining an internal cavity in communication with a bottom of the circular base through a bottom opening, the circular base including a spherical seating surface adjacent the bottom opening and a second connection structure extending above the spherical seating surface, the second connection structure configured for coupling with the first connection structure to secure the circular base to the upper body and complete the two-piece receiver;
a bone anchor comprising a longitudinal axis, a shank head at a proximal end, and an anchor portion opposite the shank head configured for fixation to the bone, the shank head including:
an upper partial spherical portion comprising an upper spherical surface having a first diameter extending downward from the planar top surface past a hemisphere plane to a circular inner edge of an upward-facing ledge; and
a lower partial spherical portion comprising a lower spherical surface having second diameter greater than the first diameter extending downward from a circular outer edge of the upward-facing ledge toward a neck portion that connects the shank head to the anchor portion;
a cap retainer having a discontinuous outer spherical surface configured to engage the spherical seating surface of the receiver and a plurality of retainer collet fingers separated by vertically-extending slots configured to resiliently expand to receive and capture the upper partial spherical portion of the shank head within the receiver when the shank head is uploaded through the bottom opening; and
a collet insert having an upper insert portion defining an insert channel configured to engage the elongate rod and upper engagement ridges extending radially outward from the upper insert portion, and a lower collet portion comprising a plurality of insert collet fingers separated by vertically-extending slots and configured to resiliently expand to receive and capture the cap retainer,
wherein the collet insert and the cap retainer are together positionable within the central bore of the upper body with the upper engagement ridges positioned within the opposed upper inner engagement grooves to maintain an initial vertical position of the collet insert and the cap retainer within the central bore, and
wherein after the cylindrical base and the upper body are coupled together to complete the two-piece receiver, a lower opening of the cap retainer is spaced above the bottom opening of the receiver.

2. The pivotal bone anchor assembly of claim 1,
wherein the first connection structure comprises a downwardly-extending annular skirt having an inward-facing annular recess formed therein, and the second connection structure comprises a plurality of flexible spring tabs having outwardly-projecting flanges at their upper ends, and
wherein the plurality of flexible spring tabs are configured to flex inward as the second connection structure is uploaded into the first connection structure, until the outwardly-projecting flanges of the circular base snap into the annular recess of the upper body to secure the circular base to the upper body and complete the internal cavity.

3. The pivotal bone anchor assembly of claim 2, wherein collet insert includes an outer cylindrical surface configured for slidable engagement the plurality of flexible spring tabs to prevent the outwardly-projecting flanges of the plurality of flexible spring tabs from disengaging from the annular recess of the upper body.

4. The pivotal bone anchor assembly of claim 1, wherein the completed internal cavity is laterally-enclosed around the circumference thereof.

5. The pivotal bone anchor assembly of claim 1, wherein after the shank head is uploaded into the lower collet portion of the collet insert, the collet insert, cap retainer and shank head are downwardly deployable together within the central bore with the tooling until the discontinuous outer spherical surface of the cap retainer engages the spherical seating surface of the receiver.

6. The bone anchor assembly of claim 5, wherein upon the downward deployment of the collet insert with tooling, the upper engagement ridges are configured to snap into opposed lower locking grooves formed into the central bore of the receiver to prevent the collet insert from moving back up within the receiver.

7. The pivotal bone anchor assembly of claim 1, further comprising a discontinuous horizontal groove formed into and extending circumferentially around the discontinuous outer spherical surface of the cap retainer and a discontinuous lower ridge located at bottom edges of the insert collet fingers,
wherein the discontinuous lower ridge is positionable within the a discontinuous horizontal groove to further secure the cap retainer within the lower collet portion of the collet insert.

8. The pivotal bone anchor assembly of claim 1, wherein the shank head further comprises an internal drive structure surrounded by the annular planar top surface and extending downward from the upper end of the shank head and configured to mate with a drive tool.

9. The pivotal bone anchor assembly of claim 8, wherein the bone anchor further comprises a shank body having an axial bore extending from the internal drive structure down to a distal end of the anchor portion and configured to receive a guide wire, the anchor portion of the shank body being configured for implantation in the bone about the guide wire with the drive tool prior to the shank head being uploaded into the central bore of the receiver.

10. The pivotal bone anchor assembly of claim 1, wherein a discontinuous annular bottom surface of the cap retainer is configured to engage an upper ledge surface of the upward-facing ledge to align the cap retainer to the shank head when capturing the shank head within the central bore of the receiver.

11. The pivotal bone anchor assembly of claim 10, wherein a diameter of the discontinuous outer spherical surface of the cap retainer is substantially equal to the second diameter of the lower spherical surface of the shank head to form a single diameter, articulating shank head sub-assembly having the second diameter that is greater than the diameter of the bottom opening of the receiver upon capturing the shank head within the cap retainer.

12. The pivotal bone anchor assembly of claim 10, wherein the cap retainer includes an annular planar upper surface that alignable flush with the annular planar top surface of the shank head upon capturing the shank head within the central bore of the receiver.

13. The pivotal bone anchor assembly of claim 1,
wherein the collet insert further comprises a pair of insert arms extending upward from a circular center portion to define the insert channel with the upper engagement ridges extend radially outward from outer side surfaces of the insert arms, and
wherein the opposite outer engagement ridges are configured to snap into the opposed upper inner engagement grooves of the central bore upon uploading of the collet insert through a lower opening of the upper body of the two-piece receiver.

14. The pivotal bone anchor assembly of claim 1, wherein the shank head is configured for axial rotation about the longitudinal axis of the shank relative to the receiver prior to locking the bone anchor assembly with the closure.

15. The pivotal bone anchor assembly of claim 1 and further comprising the elongate rod and the closure, wherein the closure is configured for positioning entirely within the central bore of the receiver above the elongate rod and in engagement with a mating structure formed into the central bore to apply a downward pressure to a top of the elongate rod, so as to secure the elongate rod to the bone of the patient.
